(19) United States
(12) Patent Application Publication
(10) Pub. No.: US 2005/0165349 A1
(43) Pub. Date: Jul. 28, 2005

(54) Needle-free injection device

(76) Inventor: Kevin Stamp, Chapeltown (GB)

Correspondence Address: SHERIDAN ROSS PC, 1560 BROADWAY, SUITE 1200, DENVER, CO, 80202, US

(21) Appl. No.: 10/767,859

(22) Filed: Jan. 28, 2004

(30) Foreign Application Priority Data
Jan. 26, 2004 (GB) 0401567.3

(51) Int. Cl. A61M 5/30

(52) U.S. Cl. 604/70

(57) ABSTRACT

A disposable gas-powered needle-free injection device comprising an outer housing having a nozzle for medicament at a forward end thereof; an inner housing located at least partly within the outer housing; a piston and ram which can be driven into the nozzle, in use, to drive medicament through the nozzle; a pierceable gas cylinder for providing driving power to the ram and piston; wherein the inner housing is axially moveable away from said nozzle, said axial movement being guided by cooperating guide means on said inner and outer housings and enabling a desired dose of medicament to be drawn into said nozzle, ready for injection.

This invention relates to the field of needleless or needle-free injection devices of the type used for injection of a predetermined dose of a medicament, for example insulin or growth hormone.

The delivery of a medicament using a needle-free injection device is typically much less traumatic than using a conventional syringe with a needle. This is because the nozzle aperture is usually of smaller diameter than a hypodermic needle and secondly because the medicament is delivered more rapidly using a needle-free injection device than by using a needle.

Needle-free injection devices, in which a piston/ram arrangement is fired forwardly to expel a liquid medicament from the device into the patient, are well-known. The piston/ram arrangement is usually powered by release of an energised mechanical spring and/or by the release of a pressurised gas. The present invention is concerned with devices which include a pressurised gas.

Single-use or disposable injection devices are desirable as they reduce the possibility of contamination. Furthermore, they may be more straightforward to use in contrast with reusable devices which usually need to be periodically dismantled in order to replace consumable parts. However, it is imperative for single-use devices to be cost-effectively manufactured.

A single-use gas-powered injection apparatus is described in WO 02/070051 (Bioject Medical Technologies, Inc) and this document also includes a useful summary of related prior art. Although directed to specific problems with technology of this type (e.g. relating to the way in which the drug is stored), WO 02/070051 also describes the basic principles of such gas-powered devices. There is provided a drug cartridge preloaded with a dose of medicament and the device is designed for single use delivery of that dose by means of a drug plunger operated by the release of a pressurised gas.

A disadvantage of devices of this type is that, depending upon the patient's requirements and the nature of the medicament being delivered, a range of differently-sized devices needs to be supplied in order to be able to deliver different doses of medicament.

Furthermore, the release of the pressurised gas determines the force with which the medicament is propelled from the device. A very sudden acceleration of the medicament may cause discomfort to particularly sensitive patients.

These issues are addressed to a certain extent by the device described in WO 00/10630(Weston Medical Limited). A needle-free gas-powered device is provided in which the stroke of the plunger can be varied by uncovering one or more holes in the wall of the device to allow the escape of some of the pressurised gas. This has the effect of allowing the force with which the injection is delivered to be reduced and for the dose to be controlled to a limited extent. However, a disadvantage of this system is that, if some of the pressurised gas is vented, not all of the medicament may be delivered into the patient and the residual quantity of the medicament will be wasted. Furthermore, the accuracy of the dose volume may be adversely affected.

It is therefore an object of the present invention to provide an injection device which seeks to alleviate the above-described problems.

According to a first aspect of the invention there is provided a disposable gas-powered needle-free injection device comprising
an outer housing having a nozzle for medicament at a forward end thereof
an inner housing located at least partly within the outer housing;
a piston and ram which can be driven into the nozzle, in use, to drive medicament through the nozzle;
a pierceable gas cylinder for providing driving power to the ram and piston;
wherein the inner housing is axially moveable away from said nozzle, said axial movement being guided by co-operating guide means on said inner and outer housings and enabling a desired dose of medicament to be drawn into said nozzle, ready for injection.

Axial movement of the inner housing away from the nozzle can be controlled so as to permit an accurate dose of medicament to be drawn into the injection device, thus reducing the risk of an incorrect dose being given and also reducing the risk of unwanted medicament being wasted.

Preferably, the device further comprises an indication of the dose of medicament which is drawn into the device.

In one embodiment, said dose indication comprises a visible scale. Alternatively, or in addition, said dose indication comprises an audible indication of the dose.

In a preferred form, said guide means comprises a substantially helical groove on said outer housing and a corresponding protrusion on said inner housing.

Ideally, said protrusion comprises a substantially helical arrangement of discrete teeth, having pits therebetween. Preferably, the device further comprises a flexible indexer tab which can ride over said teeth in order to provide said dose indication.

In a preferred form, at least one of said teeth has tapered side walls to facilitate the riding over of the indexer tab. Preferably, the rearmost one of said teeth has a substantially vertical wall which acts as an endstop over which the indexer tab is prevented from riding.

Preferably, said indexer tab is radially flexible and is located on a collar which substantially surrounds the outer housing.

The provision of an indexer tab which can ride over a series of discrete teeth means that, not only is the axial movement of the inner housing physically controllable and quantifiable by the co-operation of the teeth and tab, but the indexer tab also provides an audible click indicating the dose being loaded into the device.

In a preferred embodiment, the gas cylinder is pierceable by a piercing means, for example a spike, which is mounted within the inner housing. Preferably, said gas cylinder is forwardly-biased by a spring.

Optionally, the device further comprises a pad or seat intermediate the gas cylinder and the spring. This reduces the risk of corrosion presented by contact between adjacent metal components, as well as assisting to locate the two components with respect to one another.

Preferably, the gas cylinder is prevented from moving forward as a result of said forward bias by means of a retainer sleeve which may be fixed with respect to said inner housing.

In one embodiment, said retainer sleeve has a plurality of retention elements spaced around it which are able to move, by deformation of the material of the retainer sleeve, between a first position in which the retention elements engage with said gas cylinder so as to prevent forward movement thereof and a second position in which said retention elements spread radially out of engagement with said gas cylinder to allow the forwardly-biased gas cylinder to move towards said piercing means.

Preferably, the injection device further comprises a lock sleeve surrounding said retention elements to prevent radial outward displacement thereof, the lock sleeve being selectively axially moveable so as to release said retention elements into said second position. Ideally, said lock sleeve has apertures therein, into which said retention elements can move radially out of engagement with said gas cylinder.

In a preferred form, said retainer sleeve comprises a collet having radially-spreadable fingers, which collet in use moves between said first position in which said fingers engage with said gas cylinder and said second position in which said fingers spread radially out of engagement with said gas cylinder. Ideally, said collet fingers are biased radially-inwardly.

Preferably, axial movement of said lock sleeve is effected by depressing a button at the rear end of the injection device.

Preferably, said gas cylinder and said collet fingers are respectively provided with co-operating tapered surfaces.

In one embodiment, the ram and piston are integrally formed.

In a preferred embodiment, the rear of the ram is connected to the inner housing by means of frangible joints which, in use and upon piercing of the gas cylinder, break so as to release the ram from the inner housing. In this embodiment, said frangible joints are preferably knuckle joints suitably shaped to control the acceleration of the ram as it breaks free of the inner housing.

Alternatively, said ram and piston are freely axially moveable within the nozzle and, in use when a medicament is caused to forcibly enter the nozzle, said ram and piston move axially rearward until they abut the inner housing.

According to a second aspect of the invention there is provided a method of injecting a medicament using an injection device as described in any of the preceding paragraphs.

Preferred embodiments of the present invention will now be more particularly described, by way of example only, with reference to the accompanying drawings, in which:

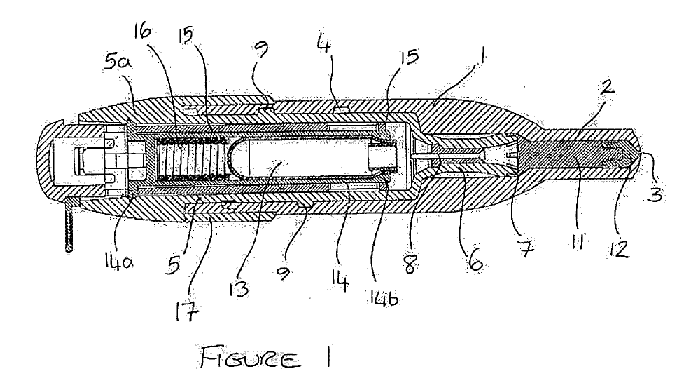

FIG. 1 is a side view of the device, shown in cross-section, in the condition as supplied to a user;

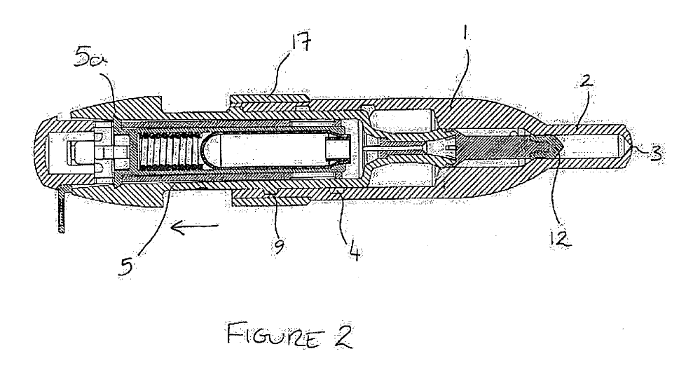

FIG. 2 is a side view of the device, shown in cross-section, showing the inner housing being rotated away from the outer housing;

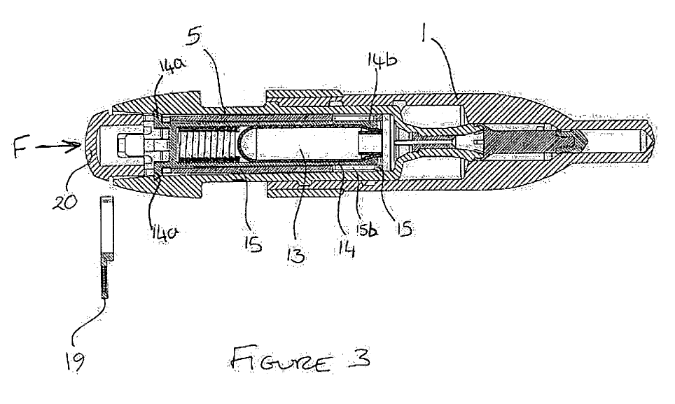

FIG. 3 is a side view of the device, shown in cross-section, with the safety clip removed and the device ready to fire;

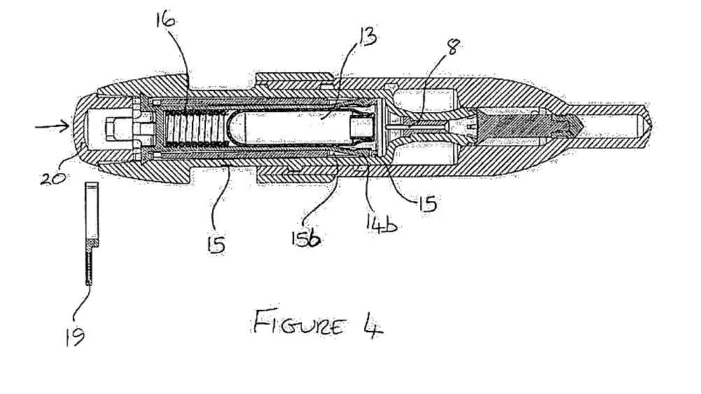

FIG. 4 is a side view of the device, shown in cross-section, immediately after pressing the button to fire the device;

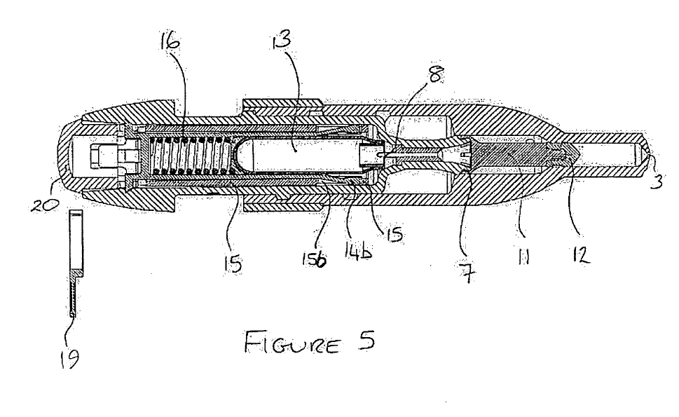

FIG. 5 is a side view of the device, shown in cross-section, at the instant of the puncturing of the gas cylinder;

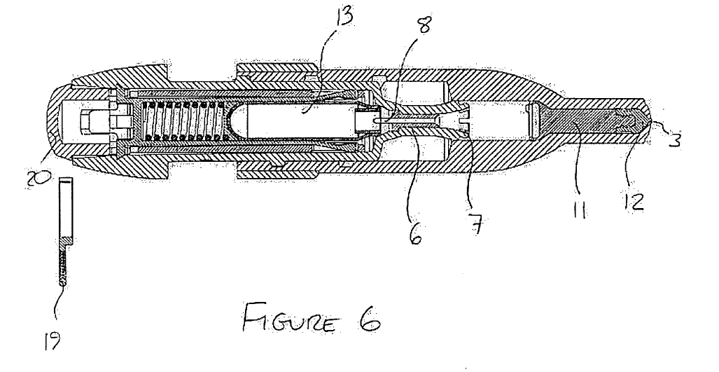

FIG. 6 is a side view of the device, shown in cross-section, immediately after delivery of an injection;

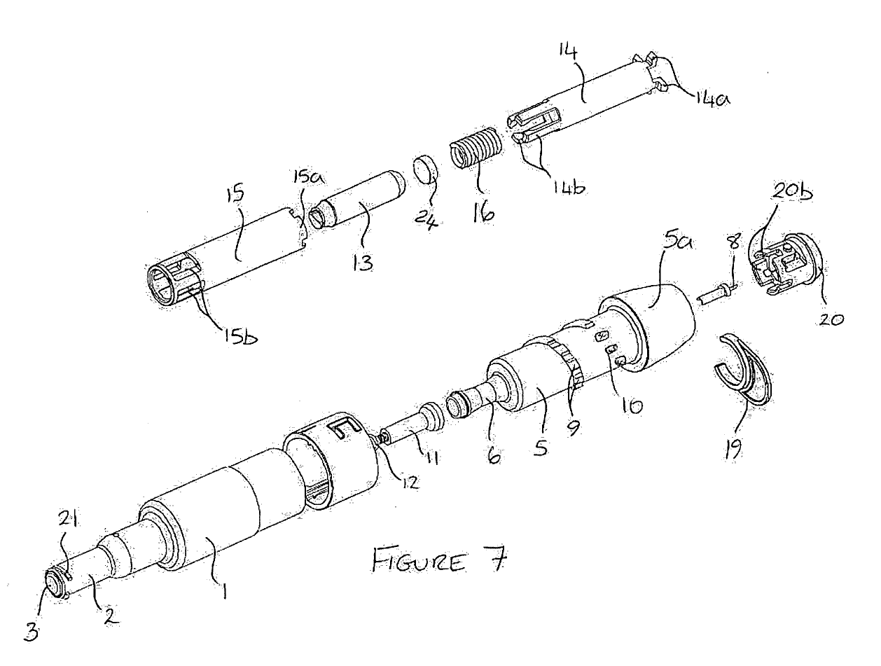

FIG. 7 is an exploded view of the component parts of the device;

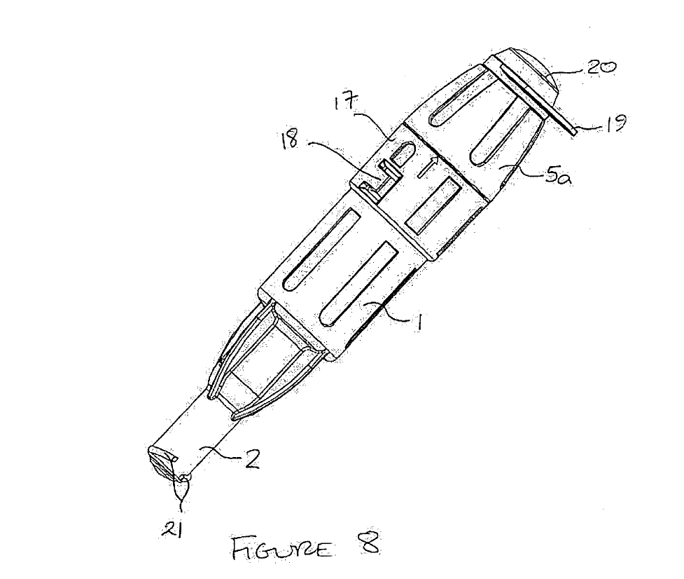

FIG. 8 is a perspective view of the device in the same condition as that illustrated in FIG. 1;

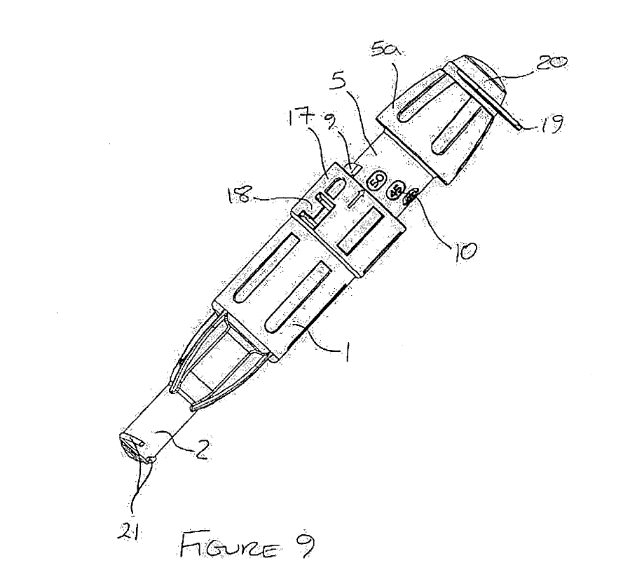

FIG. 9 is a perspective view of the device in the same condition as that illustrated in FIG. 2;

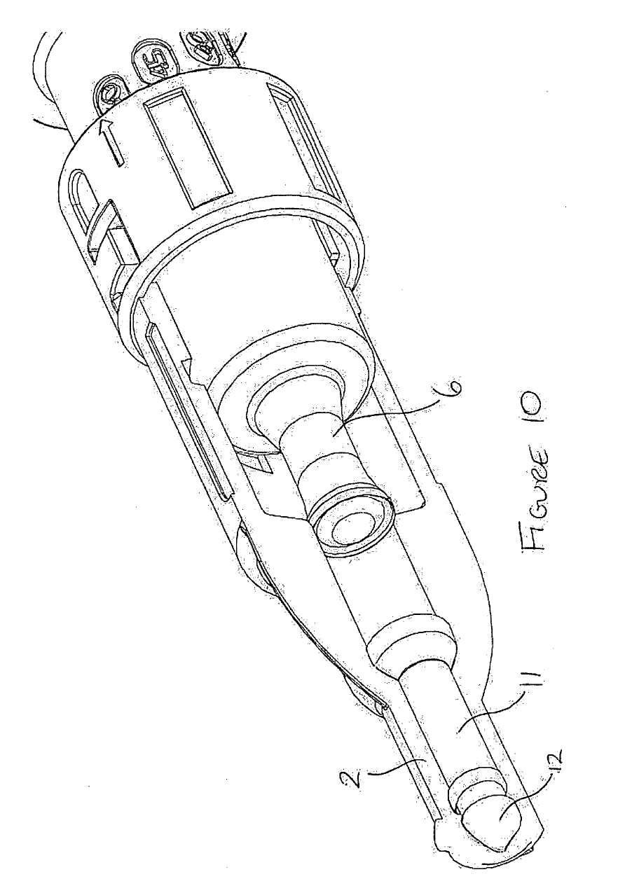

FIG. 10 is a perspective schematic view illustrating the “free-floating” ram and piston embodiment; and

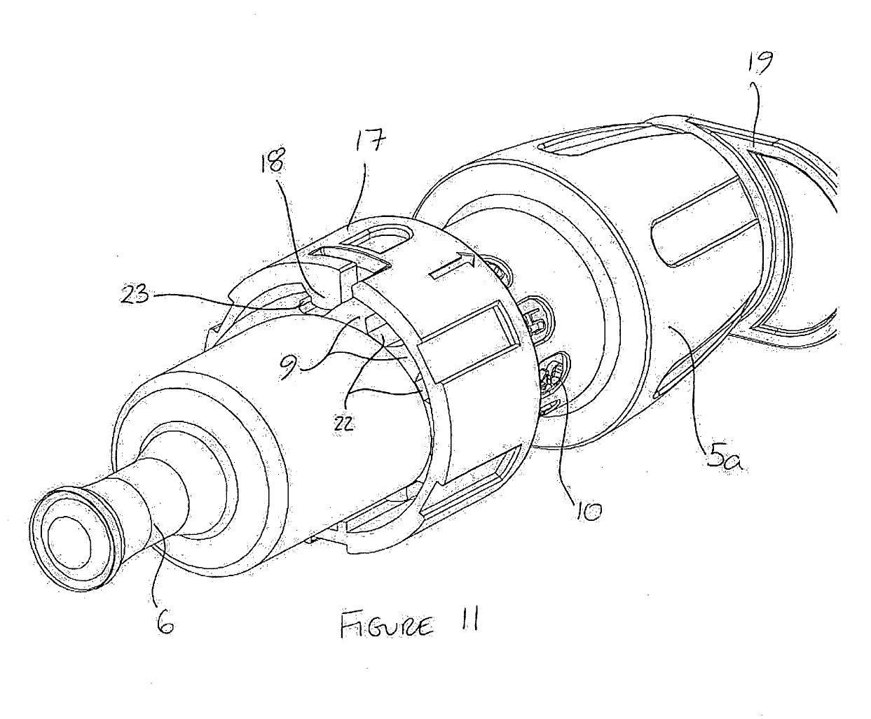

FIG. 11 is a perspective view, drawn to a larger scale, showing detail of the teeth and indexertab.

Throughout the following description, reference to a “forward” direction means the direction which is towards the patient when the injection device is in use. The “forward” end of the injection device is the end nearest the patient's skin when the device is in use. Similarly, reference to a “rearward” direction means the direction which is away from the patient and the “rearward” end of the device is the end furthest from the patient's skin when the injection device is in use.

The term “gas cylinder” means any suitable pierceable container for pressurised gas, not strictly limited to being cylindrical in shape.

The device comprises a generally cylindrical outer housing 1, having at its forward end a narrowed elongate portion 2 (the “nozzle”) with an injection orifice 3 at the end thereof. Towards the rear of the outer housing, on the inner surface thereof, there is a generally helical groove 4 which is involved in guiding the relative movement of the outer and inner housings (described later).

An inner housing 5 is provided, so-called because in normal use, most of the inner housing is situated within the outer housing 4. The forward end of the inner housing 6 is of narrowed diameter and has frangible knuckle joints 7 at the extremity thereof. Within the narrowed forward end of the inner housing is located a spike 8, firmly fixed. The spike is hollow, rather like an oversized hypodermic needle.

The central part of the inner housing is provided with a helical arrangement of protrusions or “teeth” 9 on its outer surface. The arrangement of teeth is such that, when the inner and outer housings are assembled together, the teeth 9 locate in the groove 4 on the outer housing in order to guide the movement of the inner and outer housings when the housings are rotated relative to one another. A generally helical dosage scale 10 is provided on the outer surface of the inner housing (see FIGS. 7 and 9); this scale being visible from the exterior of the device, when the inner housing 5 retreats from the outer housing 4 (as described below and illustrated in FIG. 2 et sec).

It is possible to rotate the inner housing 5 with respect to the outer housing 1 by gripping and turning the enlarged rear part 5a of the inner housing whilst holding the outer housing. This causes the overall length of the device to increase (see FIGS. 2 and 3). The enlarged rear part of the inner housing may have a textured surface or coating thereon to improve the user's grip.

A ram 11 and piston 12 are provided which fit snugly within the nozzle 2 so as to minimise dead space between the piston and the nozzle. As illustrated, the ram and piston are separate components fitted together but, alternatively, a one-piece combined ram and piston may be provided.

The rear end of the ram is attached to the forward end of the inner housing by means of the frangible knuckle joints 7. In an alternative embodiment, frangible knuckle joints are not employed; instead a “free-floating” ram and piston arrangement simply abuts the forward end of the inner housing 6. This embodiment is illustrated in FIG. 10, in which the ram and piston are shown in a forward position. When pushed backwards by a liquid medicament being forcibly loaded into the nozzle 2 through the injection orifice 3, the rear of the ram 11 abuts a seat at the front of the inner housing 6. The extent of the backward travel of the ram 11 defines the volume of the chamber into which the medicament is loaded.

Inside the inner housing 5, there is located a gas cylinder 13 which is used as the primary energy source for the device. The gas is preferably a nitrogen-helium mix, supplied in a cylinder of standard size. Differently sized injection devices according to the invention may be envisaged, containing differently sized gas cylinders.

The gas cylinder 13 is held in position within the inner housing by means of a retainer sleeve 14, the illustrated embodiment of which being referred to hereafter as a “collet”. The collet 14 has, at its rear end, a number (preferably four orthogonally-placed) flanges 14a which fix the position of the collet with respect to the inner housing. The forward end of the collet has a plurality of collet fingers 14b which grip the forward end of the gas cylinder.

The collet fingers 14b are biased radially outwardly, that is to say, they have a natural tendency to spring radially out of engagement with the gas cylinder 13. Therefore to counteract this, the collet fingers 14b are normally held or locked against the gas cylinder by means of a collet lock sleeve 15, as illustrated in FIG. 1.

A compression spring 16, located between the back of the collet 14 and the gas cylinder 13 urges the tapered front of the gas cylinder forward against the correspondingly tapered heads of the collet fingers 14b so that the gas cylinder is firmly held by the collet fingers.

A generally disc-shaped acetal pad 24 (see FIG. 7) is preferably located between the gas cylinder and the spring. The rear face of the pad 24 (which is spring-facing) is generally planar, whereas the front face (which is gas cylinder-facing) is generally concave so as to fit well with the curved rear of the gas cylinder. Not only does the pad 24 improve the fit, but also eliminates the possibility of corrosion arising from the metal gas cylinder otherwise being in contact with the metal spring.

In order to draw a dose of liquid medicament into the device, the user starts with the device in the condition illustrated in FIG. 1. The user grips and turns the enlarged rear part of the inner housing 5a whilst holding the outer housing 1. This causes the teeth 9 on the inner housing 5 to travel in the groove 4 so that the inner housing travels backwards as illustrated in FIG. 2 and the overall length of the device increases. As the inner housing travels backwards, the piston 12 retreats away from the orifice 3 in the nozzle, creating suction which draws in liquid medicament from a vial or the like attached to the front end of the nozzle (not illustrated). In the alternative embodiment in which the ram and piston are “free-floating” (FIG. 10), the liquid medicament needs to be forcibly delivered into the device through the injection orifice 3, in order to push the ram/piston backwards into the space created by the retreating inner housing.

Using the device of the present invention, it is therefore possible to load a single-use gas-powered device with a specific dose of liquid medicament. This does away with the expense of having to provide a range of disposable devices preloaded with different doses of medicament and gives the flexibility to enable the dose to be selected (in accordance with instructions) by the user.

The degree to which the inner housing retreats from the outer housing determines the volume of space available in the nozzle 2 and hence determines the dose of the liquid medicament. The user is guided whilst rotating the inner housing by a numerical or other scale 10, visible as the inner housing 5 retreats. A physical indication of the dose is also provided because the teeth 9 on the inner housing are a series of discrete teeth with pits 22 therebetween. A dose collar 17 is mounted at the rear of the outer housing 1, abutting the enlarged rear of the inner housing Sa (as illustrated in FIG. 1). The dose collar may be provided with an arrow or other visual indication pointing to the visible scale 10, as illustrated in FIGS. 9 and 11.

Referring particularly to FIG. 11, the dose collar 17 includes a downwardly depending flexible indexer tab 18 which rests between two of the series of teeth 9 on the inner housing. When the inner housing is rotated with respect to the outer housing and the dose collar, the indexer tab 18 clicks as it is forced over successive ones of the teeth 9, the number of clicks thus giving an audible and physical indication of the dose. Each of the teeth 9 has tapered sides to facilitate and guide the moving of the indexer tab 18 from one pit 22 to the next.

The inner housing 5 cannot be rotated away from the outer housing 1 indefinitely; an endstop is provided by means of the rearmost one of the teeth having a substantially vertical wall 23 rather than a tapered wall so that the indexer tab 18 cannot be easily forced thereover.

With reference to FIG. 3, with the inner housing fully retracted, the safety clip 19 can be removed so that the device is ready to fire. Before removal of the safety clip 19, the application of a downward or forward force F to the button 20 has no effect as the button 20 simply abuts the safety clip 19.

After removal of the safety clip 19, and with the device held against the patient's skin at the desired injection site, the device can be fired by applying a forward force as indicated by the arrow F in FIG. 3. The force F is transmitted by the button 20 to the collet lock sleeve 15 so that the collet lock sleeve 15 moves forward in relation to the collet 14. The collet 14 is prevented from moving forward by the flanges 14a being fixed within the housing 5. The button 20 is able to transmit force F to the collet lock sleeve 15 by cooperation of tabs 20b at the forward end of the button with tabs 15a at the rear end of the collet lock sleeve all of which fit through gaps between respective ones of the flanges 14a.

As illustrated in FIG. 3, the transmission of force F means that the collet lock sleeve 15 is now only just retaining the collet fingers 14b against the gas cylinder 13.

By pushing the button 20 further, the collet lock sleeve 15 is pushed further forward so that the collet fingers 14b spring radially outwardly into the apertures 15b provided in the collet lock sleeve 15, as illustrated in FIG. 4. At this point, the gas cylinder 13 is no longer held and the spring 16 urges the gas cylinder forward onto the spike 8 which pierces the cylinder 13, releasing the pressurised gas (see FIG. 5).

Referring to FIG. 6, the released gas travels through the narrowed front portion 6 of the inner housing, through the hollow spike 8 towards the piston/ram arrangement 11/12. The force of the gas is sufficient to break the frangible knuckle joints 7, causing the ram 11 and piston 12 to be accelerated forcefully forwards in order to eject liquid medicament from the orifice 3, thus delivering an injection. The shape of the knuckle joints 7 may be such that the acceleration of the ram and piston is controlled, in the sense that the initial acceleration is not a “hard” jolt.

Once the injection has been delivered and the device is in the condition illustrated in FIG. 6, the device can be disposed of.

FIGS. 8 and 9 are perspective views of the injection device in the same condition as illustrated in FIGS. 1 and 2 respectively.

The front portion of the nozzle 2 is provided with three short helical formations 21 onto which can be screwed a vial adaptor for holding a vial of standard size so that a dose of medicament can be loaded into the injection device as described above.

1. A disposable gas-powered needle-free injection device comprising an outer housing having a nozzle for medicament at a forward end thereof;
an inner housing located at least partly within the outer housing;
a piston and ram which can be driven into the nozzle, in use, to drive medicament through the nozzle;
a pierceable gas cylinder for providing driving power to the ram and piston;
wherein the inner housing is axially moveable away from said nozzle, said axial movement being guided by cooperating guide means on said inner and outer housings and enabling a desired dose of medicament to be drawn into said nozzle, ready for injection.

2. An injection device as claimed in claim 1 further comprising an indication of the dose of medicament which is drawn into the device.

3. An injection device as claimed in claim 2 wherein said dose indication comprises a visible scale.

4. An injection device as claimed in claim 2 wherein said dose indication comprises an audible indication of the dose.

5. An injection device as claimed in claim 1 wherein said guide means comprises a substantially helical groove on said outer housing and a corresponding protrusion on said inner housing.

6. An injection device as claimed in claim 5 wherein said protrusion comprises a substantially helical arrangement of discrete teeth, having pits therebetween.

7. An injection device as claimed in claim 6 further comprising an indication of the dose of medicament which is drawn into the device and a flexible indexer tab which can ride over said teeth in order to provide said dose indication.

8. An injection device as claimed in claim 7 wherein at least one of said teeth has tapered side walls to facilitate the riding over of the indexer tab.

9. An injection device as claimed in claim 7 wherein the rearmost one of said teeth has a substantially vertical wall which acts as an endstop over which the indexer tab is prevented from riding.

10. An injection device as claimed in claim 7 wherein said indexer tab is radially flexible and is located on a collar which substantially surrounds the outer housing.

11. An injection device as claimed in claim 1 wherein said gas cylinder is pierceable by a piercing means, which is mounted within the inner housing.

12. An injection device as claimed in claim 11 wherein said gas cylinder is forwardly-biased by a spring.

13. An injection device as claimed in claim 12 further comprising a pad or seat intermediate the gas cylinder and the spring.

14. An injection device as claimed in claim 12 wherein the gas cylinder is prevented from moving forward as a result of said forward bias by means of a retainer sleeve.

15. An injection device as claimed in claim 14 wherein said retainer sleeve is fixed with respect to said inner housing.

16. An injection device as claimed in claim 14 wherein said retainer sleeve has a plurality of retention elements spaced around it which are able to move, by deformation of the material of the retainer sleeve, between a first position in which the retention elements engage with said gas cylinder so as to prevent forward movement thereof and a second position in which said retention elements spread radially out of engagement with said gas cylinder to allow the forwardly-biased gas cylinder to move towards said piercing means.

17. An injection device as claimed in claim 16 further comprising a lock sleeve surrounding said retention elements to prevent radial outward displacement thereof, the lock sleeve being selectively axially moveable so as to release said retention elements into said second position.

18. An injection device as claimed in claim 17 wherein said lock sleeve has apertures therein, into which said retention elements can move radially out of engagement with said gas cylinder.

19. An injection device as claimed in claim 16 wherein said retainer sleeve comprises a collet having radially-spreadable fingers, which collet in use moves between said first position in which said fingers engage with said gas cylinder and said second position in which said fingers spread radially out of engagement with said gas cylinder.

20. An injection device as claimed in claim 19 wherein said collet fingers are biased radially-inwardly.

21. An injection device as claimed in claim 17 wherein axial movement of said lock sleeve is effected by depressing a button at the rear end of the injection device.

22. An injection device as claimed in claim 19 wherein said gas cylinder and said collet fingers are respectively provided with cooperating tapered surfaces.

23. An injection device as claimed in claim 1 wherein the ram and piston are integrally formed.

24. An injection device as claimed in claim 1 wherein the rear of the ram is connected to the inner housing by means of frangible joints which, in use and upon piercing of the gas cylinder, break so as to release the ram from the inner housing.

25. An injection device as claimed in claim 24 wherein said frangible joints are knuckle joints suitably shaped to control the acceleration of the ram as it breaks free of the inner housing.

26. An injection device as claimed in claim 23 wherein said ram and piston are freely axially moveable within the nozzle and, in use when a medicament is caused to forcibly enter the nozzle, said ram and piston move axially rearward until they abut the inner housing.

27. (canceled)

28. A method of injecting a medicament using an injection device as claimed in claim 1.
